(19) United States
(12) Patent Application Publication
(10) Pub. No.: US 2014/0024073 A1
(43) Pub. Date: Jan. 23, 2014

(54) BIO-MEMS FOR DOWNHOLE FLUID ANALYSIS

(71) Applicants: OLEG ZHDANEEV, Bergen (NO); BRADLEY KAANTA, Cambridge, MA (US); HUA CHEN, Yokohama (JP)

(72) Inventors: OLEG ZHDANEEV, Bergen (NO); BRADLEY KAANTA, Cambridge, MA (US); HUA CHEN, Yokohama (JP)

(73) Assignee: SCHLUMBERGER TECHNOLOGY CORPORATION, Sugar Land, TX (US)

(21) Appl. No.: 13/553,571

(22) Filed: Jul. 19, 2012

(51) Int. Cl. G01N 33/24; C12Q 1/06; G01N 27/00; G01N 27/26; G01N 7/00; G01N 25/00; G01N 27/04; C12Q 1/02; G01N 21/00

(52) U.S. Cl. 435/39; 436/28; 435/29; 435/287.1; 422/82.01; 422/82.05; 422/82.13; 422/82.12; 422/82.02; 422/82.03

(57) ABSTRACT

A method and apparatus for observing a biological microelectromechanical systems response to a fluid including combining an activator and a fluid wherein the fluid comprises a component from a subterranean formation, exposing the combined activator and fluid to a sensor in a wellbore to observe a biological microelectromechanical systems response, and integrating data from the observing with petrophysical data. A method and apparatus for observing a biological microelectromechanical systems response to a fluid including a housing comprising a biological microelectromechanical observation material, a signal analyzer in communication with the material, and a fluid preparation device positioned to allow fluid to flow to the surface, wherein the fluid comprises a component from a subterranean formation.

### FIELD

This application relates to formation fluid analysis based on a bio-MEMS sensor and/or the utilization of microbes to identify or modify formation and wellbore structures.

### BACKGROUND

Hydrogen sulfide (H₂S) generated in an oil reservoir is one of the major unwelcome impurities in petroleum. The increase of H₂S production or so-called “reservoir souring” occurs frequently in water-flooded oil reservoirs even when the reservoir is originally considered a “sweet well.” The biogenic cause of H₂S highly depends on the amount of sulfate reducing bacteria (SRB). Recent progress in microelectromechanical systems—the microelectronics, microfabrication and micromachining technologies known collectively as MEMS is being applied to biomedical applications and has become a new field of research unto itself, known as BioMEMS. The technology is originally based upon the same technology that has been used to make computer chips ever more powerful and less expensive. MEMS technology has enabled low-cost, high-functionality devices in some commonly used areas, such as inexpensive printer cartridges for ink jet printing and chip-based accelerometers responsible for deployment of automotive airbags. BioMEMS applies these technologies and concepts to diverse areas in biomedical research and clinical medicine. BioMEMS is an enabling technology for ever-greater functionality and cost reduction in smaller devices for improved medical diagnostics and therapies. the fast growth of the technology in other industries, the oil field services industry is slow to benefit from bioMEMS embodiments to analyze chemicals downhole.

Reservoir souring is characterized by an increasing concentration of H₂S in production gas. It is an example of a process that can be initiated at the microbiological level, yet can exert an effect over an entire reservoir and its produced fluids within the production lifetime of a field. The overall economic impact of microbial reservoir souring can be very significant, yet there are few technologies developed for the detection, monitoring and prevention of microbial reservoir souring. Although downhole H₂S concentration can be determined from the topsides gas phase measurement, it is desirable to in-situ monitor the generation of H₂S so that precautionary measures can be taken to prevent the reservoir souring.

It is now widely accepted that the reduction of sulfate by SRB is the most significant mechanism of H₂S generation in reservoir souring. In order to increase oil recovery by maintaining reservoir pressure and sweeping oil towards production wells, the injection of sea water and other types of water containing sulfate is a common practice. Since SRB exist widely on the earth, the injection can carry indigenous SRB to oil reservoirs. Given the abundance of residual oil and the availability of sulfate in deaerated injection water, the activities of SRB rapidly generate H₂S and increase the population of SRB.

There are multiple technologies to precisely measure the bacteria population in biological laboratories, such as automatic cell counters, flow cytometry, etc. The common working principle of these technologies is to detect the electrical/optical characteristics while biological cells are driven through an aperture/gate structure. As the cells pass through the aperture, they cause a short-term change in the impedance/intensity measurements; this change is measured as a pulse and the pulses are counted and recorded. Though this working principle is still suitable for downhole bacteria-counting detector, none of the existing devices can be used in downhole because of their dimension and working environment.

### SUMMARY

Embodiments herein relate to a method and apparatus for observing a biological microelectromechanical systems response to a fluid including combining an activator and a fluid wherein the fluid comprises a component from a subterranean formation, exposing the combined activator and fluid to a sensor in a wellbore to observe a biological microelectromechanical systems response, and integrating data from the observing with petrophysical data. Embodiments herein also relate to a method and apparatus for observing a biological microelectromechanical systems response to a fluid including a housing comprising a biological microelectromechanical observation material, a signal analyzer in communication with the material, and a fluid preparation device positioned to allow fluid to flow to the surface, wherein the fluid comprises a component from a subterranean formation. Finally, embodiments herein relate to a method and apparatus for observing a biological microelectromechanical systems response to a fluid including a sensor comprising a biological microelectromechanical observation material, a signal analyzer in communication with the material, and a fluid preparation device positioned to allow fluid to flow to the sensor, wherein the fluid comprises a component from a subterranean formation.

### FIGURES

FIG. 1 is a schematic sectional view of a wellsite and wellbore undergoing formation mixture analysis with BioMEMS.

FIG. 2 is a flow chart of two methods to implement BioMEMS systems.

FIG. 3 is a schematic diagram of an embodiment of a BioMEMs system.

FIG. 4 is a schematic view of an embodiment to construct a BioMEMS system.

FIG. 5 is a schematic view of embodiments of (a) a computer-controlled testing system; (b) an equivalent circuit model of a flowing cell in the aperture; and (c) measuring cell concentration in the micro chamber.

### DETAILED DESCRIPTION

The method and devices disclosed in the document describe an approach for chemical composition analysis of formation fluids in a downhole or surface environment, including the formation samples. Embodiments use a microfabricated biological cell-based (bioMEMS) sensor array to test certain chemicals down-hole, to provide fingerprinting, to evaluate the biodegradation level of the reservoir, acidity, metal presence, etc. This includes both hardware embodiments and methods of implementation.

This application discusses these concepts by first discussing the use of a bioMEMS chip downhole to analyze downhole fluids (salinity, PH, metal trace, etc.). This often follows the sampling-injection-conditioning-sensing-waste process. Secondly, this application discusses detecting microbes (whether injected and naturally existing in the environment) downhole to gain information of the formation properties (connectivity, biodegradation, etc.). The number, quantity, shape, and color may be observed. This information may be used to determine degradation and/or biocontaminants in the wellbore. This often follows the following process.

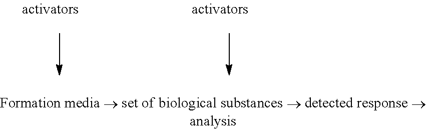

The analysis may be performed jointly while collecting and analyzing other information about a reservoir, such as petrophysical data collection and analysis. The analysis may be performed while the sensor is in the wellbore or during an oil field services treatment or both. Finally, this application briefly discusses using biomaterial as a tracing element, such as to verify connectivity between zones/wells/reservoirs or as a proxy for enhanced oil recovery effectiveness.

### Equipment Considerations

The components of the system can be grouped into several main categories:
Sample delivering system,
   microfabricated fluidic system, valves, and chambers,
   sample dilution system,
Detectors that detect both the analytical components of the modified chemicals and the florescence of the microbes,
Signal analyzers,
Software that utilizes the detector signal, controls communication between system and user, and quantitatively describes all fractions of interest.

In some embodiments, the hardware components consist of a sampling system, an injection system, a microfluidic dilution system and a biodetector array.

In some embodiments, a surface, such as a chip, may be used to analyze fluid that includes sensing conditioning, measuring and waste containment. In some embodiments, an array with sensors may be selected to ultimately provide an estimate of permeability based on comparisons of the bacteria in cells within the array (compartmentalized or complicated flow patterns may be selected to accentuate the comparisons). In some embodiments, a chip with channels may be selected wherein the channels have a selected shape, flow control or flow direction to support fluid dynamic analysis, wherein an electrostatic force is applied to provide a measuring method, wherein an ultrasonic force is applied to estimate the shape of the cells, or wherein some chemical analysis occurs.

In some embodiments, a bioMEMS cell chip module is disposed at least partially in the structural device which receives the fluid sample from the mixing dilution network of the bioMEMS preparation module into an array of cell chambers, where the fluid sample is measured and analyzed by a signal analyzer. The bioMEMS preparation module may include an injector and a mixing dilution network having one or more preparation microfluidic chambers for diluting the fluid sample into different concentrations based on a logarithmic scale. The bioMEMS or sensor package is configured for the sensor to be at least partially disposed in a high pressure environment.

In some embodiments, the bioMEMS preparation module, the bioMEMS cell chip and detection system are disposed in a device and at least a portion of the device is configured to one of secure, ensure a certain orientation, or both, the bioMEMS preparation module, the bioMEMS cell chip or detection system, when disposed into the device. A housing may encompass some or all of these components.

A processor disposed at least partially in one of the structural device, the downhole tool or both that is in communication with the signal analyzer to receive fluid sample data. The bioMEMS or the sensor package include in-situ measurements of the fluid sample while downhole in a reservoir. The in-situ measurements of the fluid sample includes testing for saturates, aromatics, resins, asphaltenes, hydrogen sulfide (H₂S), a bacteria for assessing the presence of one or more metal, a bacteria for analyzing an acidity and a salinity such as a sulfate-reducing bacteria and anaerobic fermentative bacteria.

Some embodiments may include at least one annular seal disposed between one of the bioMEMS preparation module, the MEMS cell chip module, or both and the structural device, the seal assists by providing a sealing-pressure barrier effect when the structural device is at least partially disposed in a high pressure environment. At least a portion of the structural device is configured to secure and/or ensure a certain orientation for the bioMEMS preparation module and/or the MEMS cell chip module when inserted into the structural device. At least a portion of the structural device is a recess, a protrusion, or a multi-dimensional recess or protrusion. At least a portion of the bioMEMS preparation and/or MEMS cell chip modules is structured and arranged to coordinately assist with the structure device to one of secure, ensure an orientation of or both, the bioMEMS preparation module, the MEMS cell chip module, or both within the structure device.

In some embodiments, the temperature of these chips will be fixed at the appropriate temperature depending on the biological cell, using embedded or external heaters/coolers. In some embodiments, the bioMEMS preparation module, the MEMS cell chip module, or both, have one of a thermal management device including one or more heater, one or more cooler or both. The thermal management may be one of embedded or external to the bioMEMS or sensor package.

In some additional embodiments of the invention, a downhole BioMEMS sensor may include a sampler. Before a fluid to be analyzed (referred to herein as a “formation” fluid) can be introduced into the BioMEMS system, a sample of the formation fluid may be extracted from its environment (e.g., from a rock formation in the case of boreholes). Therefore, our system may include a sampler to perform this extraction/sampling. In downhole environments, the formation fluid may be at high pressure (e.g., up to 30 kpsi) and high temperature (up to 200 C, or even higher). The HPHT sample may be depressurized before being analyzed thru the sampler/injector.

Once adjusted for pressure, the sample may be conditioned by mixing with conditioner solutions. This may include dyes, buffer solutions, etc. Reservoirs of these conditioning solutions may be part of the downhole system implementation. Some embodiments may have only one reservoir or several reservoirs depending on the number of fluids required. These fluids are mixed with the sample fluid inside of a mixer. Fluid ratios are precisely controlled by the mixer.

One embodiment of the mixer may be in the form of a section of flowline, where the injected fluids get mixed over a certain length of the tube. Another embodiment of the mixer may be in the form of a chamber bottle. Yet another embodiment may involve a micro-machined MEMS chip with pre-etched channels. The fluid resistance of these channels will determine the mix ratio of fluids. The temperature of the chip can be precisely controlled by embedded heating devices (heaters) and cooling devices (Peltier devices).

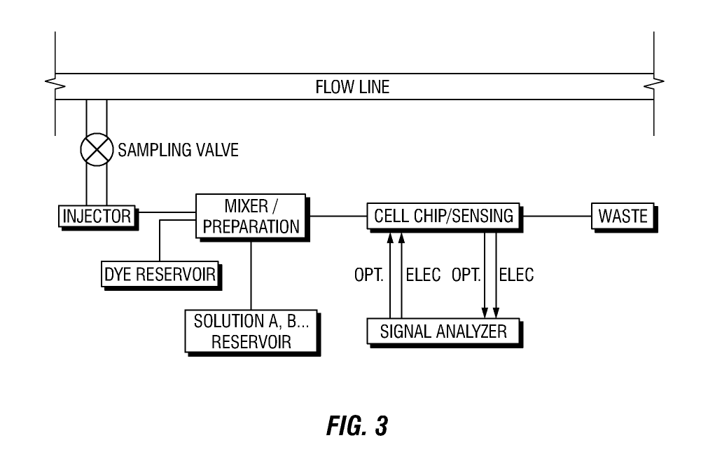

In any event, the biosensor system may contain two separate modules: a MEMS sample preparation chip for pH and osmolarity adjustment, and a MEMS cell chip for dilution and cell growth, as shown in FIG. 3. According to one embodiment, a downhole BioMEMS sensor may include a sampler. Before a fluid to be analyzed (referred to herein as a “formation” fluid) can be introduced into the BioMEMS system, a sample of the formation fluid may be extracted from its environment (e.g., from a rock formation in the case of boreholes). Therefore, the system may include a sampler to perform this extraction/sampling. In downhole environments, the formation fluid may be at high pressure (e.g., up to 30 kpsi) and high temperature (up to 200 C, or even higher). The HPHT sample may be depressurized before being analyzed through the sampler/injector.

FIG. 3 illustrates one embodiment of the following mechanical, chemical and biological components. A bioMEMS preparation module includes an injector and a mixing dilution network having one or more preparation microfluidic chambers for diluting the fluid sample into different concentrations based on a logarithmic scale.

The bioMEMS preparation module, the MEMS cell chip module, or both, may have at least one detector that detects one or more properties of the fluid sample. In some embodiments, the detector is one of a microbial detector, a pressure sensor, a temperature sensor, a pH sensor, an osmolarity sensor, an optical sensor, a resistivity sensor, a density sensor, a viscosity sensor or a capacitance sensor. The bioMEMS or sensor package may include an interrogation device, the interrogation device is one of an optical device, non-liner optical device, an electrical device, or both. The bioMEMS or sensor package is manufactured from silicon, silicon oxide, quartz, sapphire, glass, metal, PEEK or some combination thereof.

Some embodiments may include a plurality of biosensors coupled to the bioMEMS cell chip module and the bioMEMS preparation module, the plurality of biosensors including an organic sensor, an inorganic ion sensor, an electrochemical sensor, an inorganic ion sensor, a chemical sensor, a small molecule sensor, a cell sensor, a bacteria sensor, a pressure sensor, a temperature sensor, a pH sensor, a osmolarity sensor, an optical sensor, a resistivity sensor, a density sensor, a viscosity sensor, or a capacitance sensor.

In some embodiments, a micro downhole bacteria-counting detector for determining the biogenic generation rate of H₂S is used. It consists of 3-D silicon-based microfluidic channels and chamber and electrode pairs for admittance measurement. The proposed detector will be able to in-situ and instantly read out the SRB concentration of downhole fluid sample.

The bacteria-counting detector consists of the following features: two apertures for electrical detection, microfluidic chambers and channels for sampling, and electrical connection circuitries. Dimension of the apertures is highly critical for the cell counting because it has to fulfill the following functions: 1) allowing only one bacterium going through at one time; and 2) providing optimal space for impedance sensing. Finite element analysis of electrical field will be employed in the design of apertures. Volume of the micro chamber and configuration of the microfluidic channels will affect on the operation parameters such as flow rate and sampling volume, so that microfluidic simulation will also be performed.

After the mixer, the fluid is introduced to the analyzer chip. The purpose of this BioMEMS chip is to interrogate the fluids via the presence and interaction of microbes with the fluid being analyzed. Various of method and mechanism can be leveraged to interrogate the cell response. These include, but not limited to, optical and electrical signals. In some embodiments, the fluid after analysis gets dumped into a waste chamber.

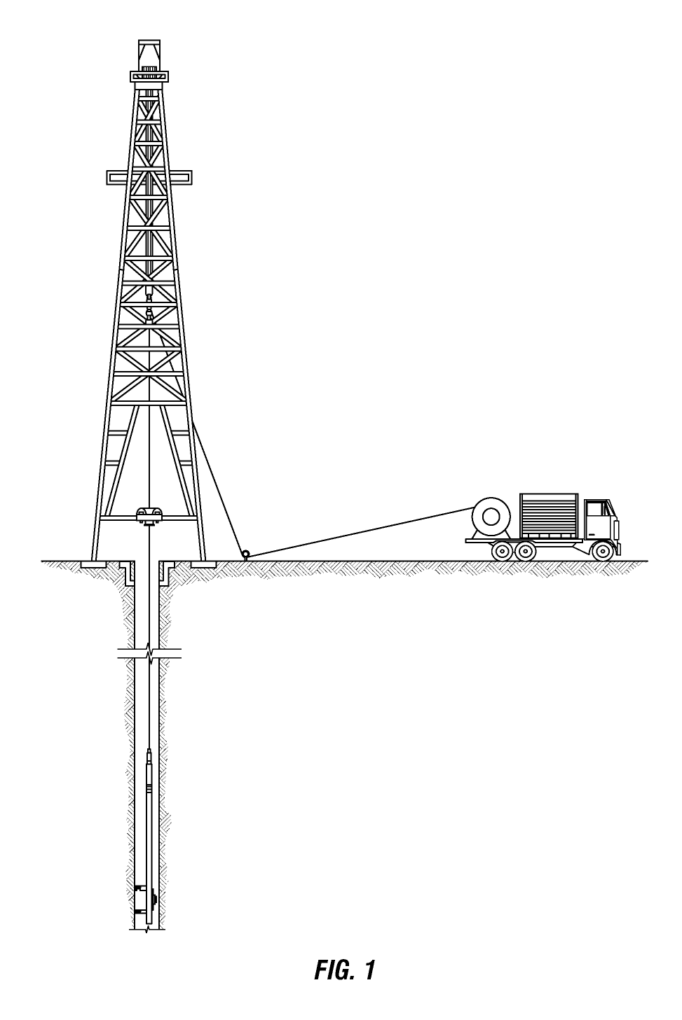

Next, one implementation of BioMEMS for in-situ formation fluid analysis is discussed. Initially referring to the diagram in FIG. 1, a small quantity of the formation fluid is extracted from a reservoir using a sampling tool. Then the formation fluid, after preliminary filtering, (e.g., to remove sand particles) via a sampling tool flowline is delivered to the module where a bioMEMS system is placed. The sample of formation fluid is injected into the separation module. In one embodiment of the tool, there are multiple separation modules in an arrayed format, which are isolated and used only for one analysis each.

In some embodiments, a bioMEMS cell chip module disposed at least partially in the structural device receives the fluid sample from the mixing dilution network of the bioMEMS preparation module into an array of cell chambers, wherein the fluid sample is measured and analyzed by a signal analyzer, wherein the bioMEMS or sensor package is configured for the sensor to be at least partially disposed in a high pressure environment, for oilfield related applications including oil and gas, or both. The bioMEMS preparation module includes one or more conditioning microfluidic chambers having at least one conditioning fluid. The microfluidic control channel is in communication with a downhole tool that is in communication with a reservoir. One filter in communication with the fluid sample is positioned in one of the structure device or the downhole tool, to remove one or more particles in the fluid sample having a size larger enough to block the microfluidic control channel, at least one phase of interest or some combination thereof. In some embodiments, the filter my selectively filter water, oil, gas, mud solids, or a combination thereof.

Once adjusted for pressure, the sample may be conditioned by mixing with an activator, such as a conditioner solution. This may include dyes, buffer solutions, etc. Reservoir of these conditioning solutions may be part of the downhole system implementation. These fluids are mixed with the sample fluid inside of a mixer. Fluid ratios are precisely controlled by the mixer. Further, in some embodiments, the activator includes a material selected to react with a subterranean component such as an acid, a base, a food source, a conditioner, a reproductive inhibitor, a biocide or a combination thereof.

One embodiment of the mixer may be in the form of a section of flowline, where the injected fluids get mixed over a certain length of the tube. Another embodiment of the mixer may be in the form of a chamber bottle. Yet another embodiment may involve a Micro-machined MEMS chip with pre-etched channels. The fluid resistance of these channels will determine the mix ratio of fluids. The temperature of the chip can be precisely controlled by embedded heating devices (heaters) and cooling devices (Peltier devices).

After the mixer it is the analyzer chip. The purpose of this BioMEMS chip is to interrogate the fluids via the presence and interaction of microbes with the fluid being analyzed. Various of method and mechanism can be leveraged to interrogate the cell response. These include, but not limited to, optical and electrical signals. In some embodiments, the fluid after analysis gets dumped into a waste chamber.

The microfluidic valves are actuated either pneumatically or electrostatically, for both modules. Initially, each water sample is loaded into the preparation chamber, while cells are loaded into the cell chamber. The osmolarity and pH of the water samples are adjusted, and then driven towards dilution network. This network dilutes the sample into several different concentrations on a logarithmic scale. Finally, these diluted samples reach the cell chamber during a certain assay period. After the assay incubation is complete, the cells are measured, e.g. by an external fluorescent microscope. The fluid inside the chamber after incubation can be analyzed for more information.

The conditioning fluid of the one or more conditioning microfluidic chambers is mixed with the fluid sample in the one or more preparation microfluidic chambers of the bioMEMS preparation module to condition the fluid sample. The conditioning of the fluid sample includes adjusting one of pH, osmolarity, or both. The conditioning fluid may contain a trace fluid, at least one bacteria, an activation reagent used for stimulating a bacteria activity to transform the bacteria activity from a sleeping mode to an active mode.

### Manufacture of the Equipment

One embodiment of these chips is made by micromachining Silicon, glass and soft materials are commonly used in MEMS and bioMEMS. The connection among these MEMS chips could, in one embodiment, be realized thru a micro-fluidic platform, which couples the injector, the mixer, the analyzer and the waste. Thermal management of each section can be handled independently if needed. The microfluidic platform includes micro-channels for the flow paths of the fluid sample and conditioners. The microfluidic channels may be constructed by a variety of techniques. For example, silicon-glass substrates containing microchannels may be anodically n=bonded to encapsulate complex fluid circuits that communicate with components. The microfluidic channels may be etched using, for example, lithography-based techniques known in the art. Micro-fabrication techniques may allow positioning of components over the microfluidic platform with tolerance within a few microns such that the fluidic ports are well aligned with a high degree of surface flatness. Either anodic bonding, or other bonding techniques, or O-rings can be used to achieve sealing.

Fabrication:

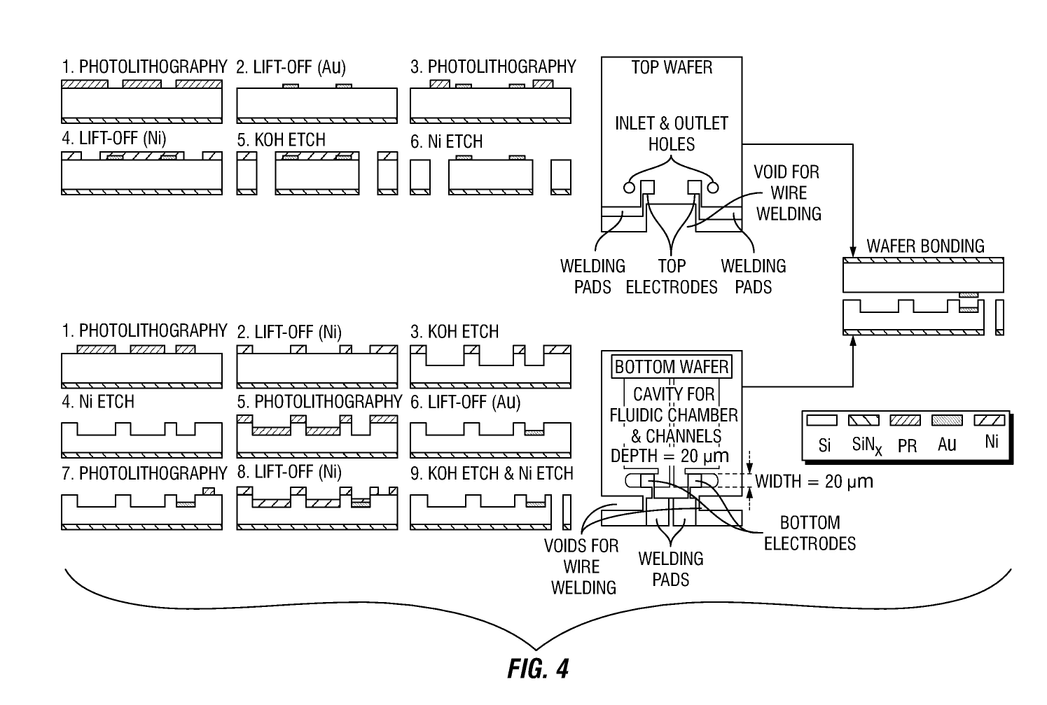

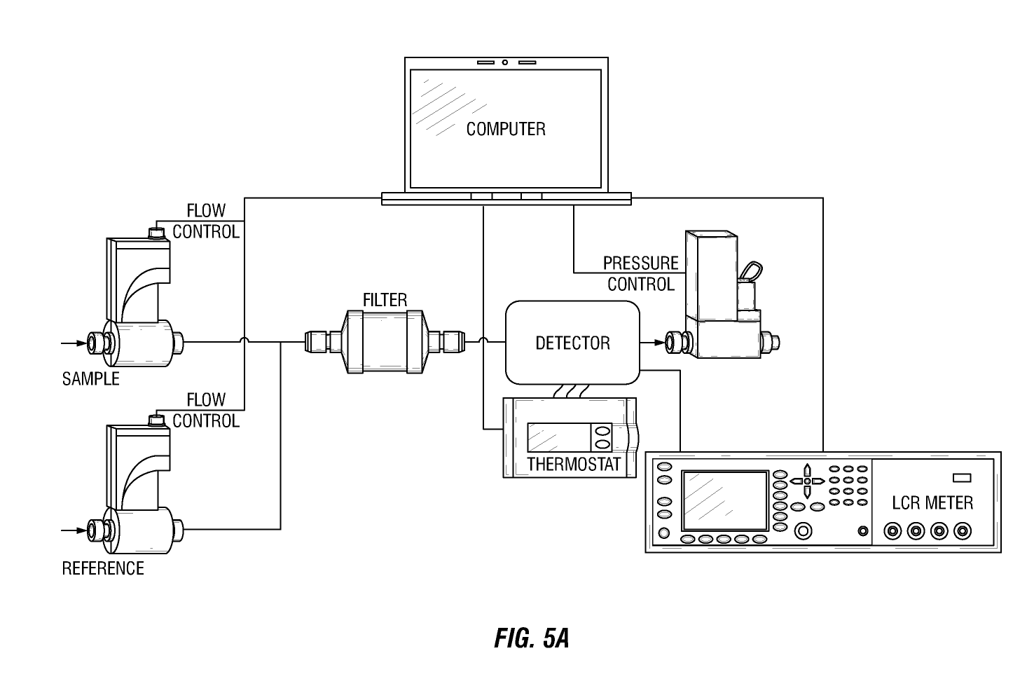

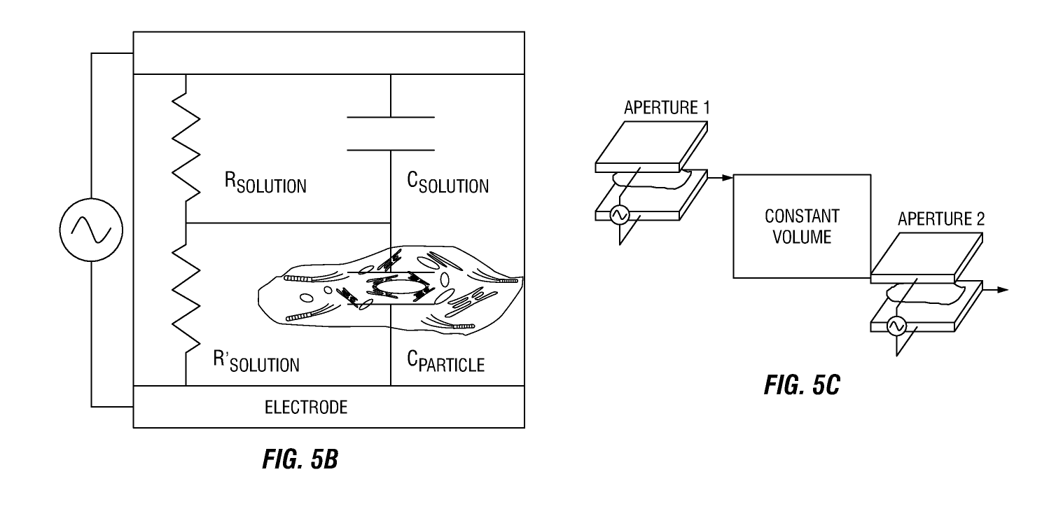

The detector will be fabricated on commercial double-side polished (110) silicon wafers precoated with silicon nitride (SiNx) on one side. Generally, the fabrication processes will be divided into two parallel paths for top wafer and bottom wafer respectively. The impedance sensing electrodes will be made of gold in electron beam evaporation so that highly smooth surface can be achieved. In order to create the microfluidic chamber and channels in bulk silicon, potassium hydroxide (KOH) etch will be employed due to its extremely anisotropic characteristics. Correspondingly, nickel layer prepared in electron evaporation will be used as the mask material while it will be removed in chemical Ni etch after the KOH etch. The same etching processes will be also used to create those through-wafer voids for liquid inlet/outlet and wire welding. Eventually, the top and bottom wafers will be bonded together after a thorough cleaning of the bonding surface. The microfabrication processes are shown in FIG. 4.

The quality control of fabrication technologies is critical to the performance of bacteria-counting detectors. Especially, since the surface roughness of gold electrodes will remarkably influence the inductance value on the liquid/metal interface, uneven profile of layers can lead to serious inconsistency of sensor performance. Secondarily, the anisotropic KOH etch will decide the surface profile of apertures, affecting the impedance sensing. Along with the fabrication processes, several characterization technologies, such as AFM, SEM and XPS, will be employed for monitoring the quality of microfabrication processes, i.e., thickness and surface morphology. The record of coating quality will be used as reference in the sensor characterization.

Modularization and Package:

The bacteria-counting detector will be integrated into a module in order to fit into a downhole tool string. In order to carry out in-situ SRB detection downhole, the bacteria-counting module contains two major functional parts which are fluidics and electronics sections, as shown in FIG. 3. In the fluidic section, the delivery of the downhole fluid sample to the detector will be pre-filtered via some membrane separation technique in an implanted microfilter. The microfluidic channels will be made high-pressure compatible. A package process will be developed to provide electronic and fluidic connection for the bacteria-counting detector. In order to enhance the electronic signals for transmission, a preamplifier will be included into the module. The regulation of valve functioning, magnification of electrical signal and electrical communication will be all carried out through the electronic section integrated in the module.

Material Selection:

One embodiment uses silicon and glass to build the MEMS chip and since the fabrication process goes thru 400 C, downhole temperature (usually up to 200 C) is not a problem. The electrical signals are passed thru deposited conducting paths made from metal (typically Pt or Au) or doped Silicon. These are HT compatible as well. Materials like PDMS that are commonly used in pharmaceutical and healthcare are not compatible with downhole temperature. Compatibility with the microbes and the fluids needs to be considered as well.

According to another embodiment, the microfluidic platform may be manufactured out of metallic substrates that may be bonded by thermal diffusion. The micro fluidic pathways within the substrate may be molded or machined by micro-EDM (electric discharge machining) process. Other manufacturing options may also be used to construct the microfluidic platform. Other manufacturing techniques may eliminate the use of tubing and related connectors.

MEMS Packaging and Ruggedization:

Embodiments of the bio-MEMS chip described herein may need to be packaged and ruggedized to protect the chip from hostile downhole environment, esp. shock and vibration. Different techniques, including chip level protection using multilayer of silicone/glass/peek/other soft materials and chip-to-world mechanical packaging to absorb shocks. This also includes how other fluids are introduced to the system to aid analyses, such as the dyes and other reagents. The packaging also includes the interrogation means, including optical and/or electrical signals.

The structure of the bioMEMS chip would need to be ruggedized, in order to function in harsh downhole environment (HPHT, corrosion). This includes material selection (HT-200 C) for the chip and its packaging, anti-corrosion coating, MEMS packaging, ruggedizing to survive shock (250 g).

Anti-Corrosion Coating:

Thin-layer coating materials are often used to fight corrosion which would be a big problem in downhole conditions, with both the corrosive fluids (high salinity water and H2S) and high temperature that accelerates corrosion. These coatings range from commercially available coating services to less common ones like Atomic Layer Deposition, or ALD, where dense layers of atoms are deposited to form an anti-corrosion coating. The bioMEMS or sensor package includes at least one coating, such as an anti-corrosion coating, a hydrosulfide protection coating, a mercaptans protection coating, a surface finish protection coating or another device protection coating.

Reliability:

The harsh downhole environment will be simulated in laboratory for the reliability test of our bacteria-counting detector. High temperature up to 200° C. and brine/acid immersion will be applied to the bacteria-counting detector for certain period of time to evaluate their lifetime in harsh environment. Failure analysis will be performed to the sensors tested in order to discover potential flaws existing in the design and fabrication processes. Once standard lifetime of the bacteria-counting detector is determined in the failure test, consistency of the bacteria-counting detector within their lifetime will be evaluated in repeated detector characterization processes. The result of consistency test will be one of the crucial features in establishing the standard of manufacturing quality control.

### Method Considerations

Generally, the method and apparatus herein may be used in combination with petrophysical data to estimate the following properties or to enhance the estimates provided by petrophysical data for porosity, permeability, composition, pressure, viscosity, density, pH, resistivity, dielectric constant or a combination thereof. The composition may include hydrocarbon content, trace element content, biomarker content or a combination thereof. The hydrocarbon content may include saturate, aromatic, resin, asphaltene, or a combination thereof.

Formation fluid analysis to evaluate the level of biodegradation in-situ is desirable. In addition, implementation of the complementary bulk properties measurements to optical analysis that have a limited ability to resolve the presence of different components in a complex mixture will allow us to elevate the level of confidence in the sample analysis and uncover its complex nature.

Embodiments may be configured to provide the following functions.
fingerprinting of oils and water aquifers based on a microbiological pattern.
evaluating reservoir salinity/acidity.
evaluating the presence of heavy metals.
evaluating the biodegradation level.
evaluating the gas source: biogenic vs. thermogenic.
evaluating carbon isotope ratio.
identifying reservoir heterogeneity based on microbiological pattern.
water/gas breakthrough zonal isolations using the biological plug.
creation of high permeability zones in the perforation/formation.
trace analysis based on microbe injection.
sampling heavy oil by reducing the viscosity by biological reaction.
modifying wettability of the formation by microbe injection.
ensuring chain of custody by biological means.
utilizing bio-MEMS sensor for chromatography/spectrometry.
utilizing bio-MEMS sensor for specific chemicals detection.

In some embodiments, the in-situ measurements of the fluid sample include in-situ measurement data, the in-situ measurement data is stored on at least one processor for analyzing the fluid sample one of a level of biodegration in-situ, a fingerprinting of a microbial pattern, an evaluation of a biodegradation level of the fluid sample over a periodic time period, a compartmentalization of a property, a chemical or both of the fluid sample, a non-biological reaction of one or more elements of the fluid sample, identifying a transformation or a chemical process of an activity of a stimulated grow or rapid degradation of one or more compounds in the fluid sample, cells or bacteria in the fluid sample.

The one or more sensors may measure properties of the fluid in a subterranean environment, such as hydrogen sulfide (H₂S) concentration, a first bacteria that assists in assessing one or more type of a metal or a bacteria that assists in analyzing an acidity and a salinity such as a sulfate-reducing bacteria and anaerobic fermentative bacteria.

Some methods may benefit from using a downhole tool to obtain a fluid sample from a wellbore in the subterranean environment, communicating the fluid sample from the downhole tool to a bioMEMS preparation module disposed at least partially in a structural device within the downhole tool, conditioning the fluid sample with one or more conditioning fluid while in the bioMEMS preparation module, and communicating the fluid sample from bioMEMS preparation module to a bioMEMS cell chip module disposed at least partially in the downhole tool, wherein the fluid sample is measured and analyzed by a signal analyzer that is at least partially disposed in a high pressure environment.

Bacteriological analysis as described herein allows an in-situ selective marker-free identification of formation sample. The results of the proposed analysis can complement the existing optical methods and future analytical characterization apparatus.

### H₂S Detection

Biological cell-based MEMS can be extremely sensitive to the presence of H₂S, given the right type of biological cell.

### Metal Presence

It has been suggested that reduction of iron is an ancient and widespread mechanism for anaerobic respiration of thermophilic and hyperthermophilic microorganisms in deep subsurface petroleum reservoirs. By measuring the chemicals before and after interaction with the biological cells in the MEMS chambers, or by measuring the signals from the bacteria, the presence of certain metal like iron can be detected.

### Acidity and Salinity

“Souring” of oil reservoirs by the formation of hydrogen sulfide has been a problem since the beginning of commercial oil production. Sulfate-reducing bacteria were found widespread in oil-well production waters. Some oil reservoirs contain highly saline brines with salinity above 20%. Anaerobic fermentative bacteria that can grow at this high salinity have been isolated from such oil wells in Africa, the Gulf of Mexico and USA. By using these sulfate-reducing bacteria and anaerobic fermentative bacteria, the acidity and salinity of the reservoir can be analyzed.

### Biodegradation Level of the Reservoir

Biodegradation is generally recognized as a series of natural processes resulting from the activities of microorganisms, by which organic materials are converted to simpler compounds and finally to inorganic substances (CO₂, CH₄, H₂O, NO₃⁻, SO₄⁻, PO₄⁻, etc.). Biodegradation often (but not always) involves utilization of substance being broken down as a source of carbon-energy by the degrading organism. The rate at which a given compound will degrade when placed in certain natural environment is determined by many factors which interact in a complex manner. Some of these reactions will be non-biological in nature. Only few chemicals are completely non-reactive in the environment, i.e., their concentrations change only as a result of physical processes of dilution and dispersion. Most chemicals undergo a variety of transformations (particularly in an aqueous environment), such as acid-base, complexation, oxidation-reduction, sorption, hydrolysis, photolysis, and biotransformation.

pH

The pH value of a growth medium is an important parameter which effects the rate of many types of reactions not only the ionization of acids and bases but also oxidoreduction reactions in the cell.

### Carbon Source

When the sole carbon and energy source in the medium is changed the redox-potential within the cell can change and this results in changes in the carbon-flux within the cell.

To investigate the range of carbon sources which can support growth, on receipt, each water sample shall be analyzed to determine pH, total organic carbon (TOC), volatile fatty acid (VFA) and sulphate levels. The pH of the waters shall then be adjusted to a certain range, e.g., 6-8, and VFA added to give waters of high and low VFA levels.

### Optical Signals

Optical observation using a camera image can be used to identify the different colony shape, size, color and texture (dry or wet), colored pictures of colony shapes should be provided. Alternatively, optical transmission/absorption at different wavelength could also provide useful information of the cells.

### Motility and Activity Test

### Electrical Signals (Resistivity, Dielectric Constant, Etc.)

### Biotyping of the DNA/RNA's

### Permittivity

According to the specific requirement related to downhole environment, the desired detector will be based on silicon material that can be processed in microfabrication technologies. Besides, the downhole fluid sample may contain particles of which dimensions are close to those of bacteria. It will cause the difficulties to distinguish them with the conventional bacteria-counting technologies. Therefore, in the desired detector, the permittivity measurement of particle will be carried out to identify bacteria from the downhole fluid sample.

### SRB Population Monitoring

The generation of H₂S is results of SRB metabolism in which the environment temperature and the supply of sulfate are the key rate-controlling features. Given the total sulfur amount can be detected before the injection of water and the environment temperature can be measured downhole, the monitoring of SRB population will give us the concentration and biogenic generation rate of H₂S. It will facilitate the monitoring of crude petroleum quality, as well as the prevention of reservoir souring through the online assessment of biocide efficiency in downhole.

### Bacteria-Counting Detector for Monitoring Downhole Biogenic H₂S

Some embodiments will also benefit from a downhole bacteria-counting detector and to characterize the responses of detectors to individual micro particle species.

### Characterization

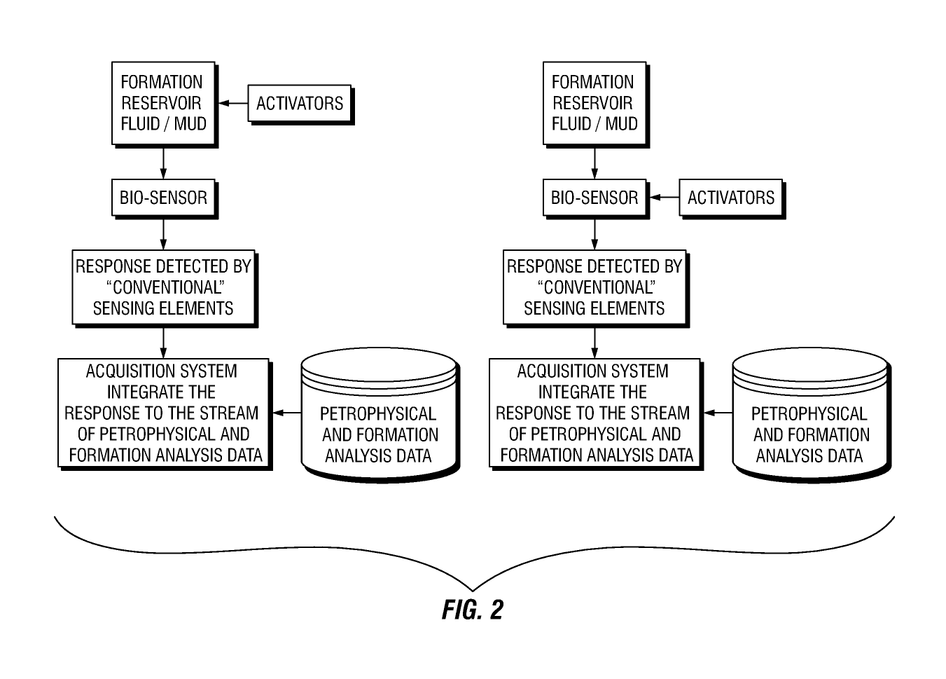

A computer-controlled electrical measurement system will be set up to read out impedance sensing results of the detector using a LCR meter (FIG. 2a). This may be used to characterize regions of a formation. The downhole fluid sample supplied with the flow controls will first go through a filter to remove all particles that is possible to block the microfluidic channels. In order to distinguish other inorganic particles, such as SiO₂ and Al₂O₃, from bacteria, an AC electric field will be applied to aperture space between the gold electrodes. Given these inorganic particles have relatively low permittivity (<10∈₀ at 10 kHz) and bacteria have relatively high permittivity (˜100∈₀ at 10 kHz), it is feasible to exclude the inorganic particles for the bacteria amount. Therefore, impedance spectroscopy of the static flow experiments with or without particles in the aperture will be conducted to determine an equivalent circuit model (FIG. 2b) in which the particle is mainly considered a dielectric body. The applied electric field frequency will be also optimized for the best distinguish of bacteria while the reasonable flow rate will be matched to this frequency. The cell counting mechanism is shown in FIG. 3c. Aperture 1 will record the inlet cell amount while the outlet cell amount will be measured by Aperture 2. When difference between the recordings of two apertures becomes stable, it will be considered the cell amount in the constant volume of micro chamber, leading to the result of cell concentration.

To manipulate the cells in the chamber, various methods could be used. These include but not limited to the following:
Electrostatic field and force: one could apply voltage across the microfluidic channels to capture/manipulate the cells.
Ultrasonic field and forces. One could use ultrasonic field to lock cells in certain locations in the flow.
Thermal gradient.
Fluid dynamics.
Chamber structure and flow restriction. Various flow paths and restriction could be utilized and switched to manipulate the flow direction and rate, which carries the cell. For example, one could construct chambers that is easy for certain size bacteria to enter and very difficult to exit. Then the next cell will be captured in the next chamber in the flow path and so forth. By detecting optical/electrical signals from the cells, one could count the numbers of a given type of bacteria.

### ADVANTAGES

Therefore, the capability to in-situ determine the SRB population will be particularly valuable in monitoring the reservoir souring. Furthermore, the SRB population obtained in continuous monitoring can be used to calculate the metabolism rate of SRB, leading to the quantitative measurement of biogenic generation rate of H₂S.

The continuous measurement of SRB concentration can be used to evaluate the metabolism rate of SRB so that we can calculate the H₂S generation rate. As compared to the state of the art, our bacteria-counting detector exhibits the following advantages: (1) measuring dynamically changing rate; (2) solid and simple structure; (3) anticorrosion against brine and acids; (4) compact configuration without moving parts; (5) instant electrical detection; (6) convenient in-situ operation. Successful delivery of the proposed milestones will lead to a dramatic improvement of downhole fluid analysis.

We claim:

1. A method for observing a biological microelectromechanical systems response to a fluid, comprising:
combining an activator and a fluid wherein the fluid comprises a component from a subterranean formation;
exposing the combined activator and fluid to a sensor in a wellbore to observe a biological microelectromechanical systems response; and
integrating data from the observing with petrophysical data.

2. The method of claim 1, wherein the integrating occurs while the sensor is in the wellbore.

3. The method of claim 1, wherein the integrating occurs while an oil field service is occurring in the wellbore or formation or both.

4. The method of claim 1, wherein the combining occurs before combined activator and fluid are exposed to the sensor.

5. The method of claim 1, wherein the combining occurs while exposing the activator and fluid to the sensor.

6. The method of claim 1, wherein the petrophysical data is selected from the group consisting of porosity, permeability, composition, pressure, viscosity, density, pH, resistivity, dielectric constant, or a combination thereof.

7. The method of claim 6, wherein the composition comprises hydrocarbon content, trace element content, biomarker content, or a combination thereof.

8. The method of claim 7, wherein the hydrocarbon content comprises saturate, aromatic, resin asphaltene, or a combination thereof.

9. The method of claim 1, wherein the activator comprises a material selected to react with the component.

10. The method of claim 9, wherein the material is selected from the group consisting of an acid, a base, a food source, a conditioner, a reproductive inhibitor, a biocide, or a combination thereof.

11. The method of claim 1, wherein the sensor comprises a biological indicator.

12. The method of claim 11, wherein the indicator is a microbe.

13. The method of claim 11, wherein the biological indicator is bacteria.

14. The method of claim 1, wherein the integrating comprises estimating the population of sulfate reducing bacteria.

15. The method of claim 1, wherein the integrating comprises identifying reservoir compartmentalization.

16. An apparatus for observing a biological microelectromechanical systems response to a fluid, comprising:
a housing comprising a biological microelectromechanical observation material;
a signal analyzer in communication with the material; and
a fluid preparation device positioned to allow fluid to flow to the material, wherein the fluid comprises a component from a subterranean formation.

17. The apparatus of claim 16, further comprising a valve between a flow line and the device, wherein the flow line comprises the component.

18. The apparatus of claim 16, wherein the housing comprises a dye reservoir.

19. The apparatus of claim 16, wherein the housing comprises a solution reservoir.

20. The apparatus of claim 19, wherein the solution reservoir contains a fluid selected from the group consisting of an acid, a base, a food source, a conditioner, a reproductive inhibitor, a biocide, or a combination thereof.

21. The apparatus of claim 16, wherein the material comprises a biological indicator.

22. The apparatus of claim 21, wherein the indicator is a microbe.

23. The apparatus of claim 21, wherein the indicator is bacteria.

24. The apparatus of claim 16, wherein the housing comprises a waste collection region.

25. The apparatus of claim 16, wherein the signal analyzer comprises optical observations.

26. The apparatus of claim 16, wherein the signal analyzer comprises electrical observations.

27. The apparatus of claim 16, wherein the device comprises a filter.

28. The apparatus of claim 27, wherein the filter selectively filters water, oil, gas, mud solids or a combination thereof.

29. An apparatus for observing a biological microelectromechanical systems response to a fluid, comprising:
a sensor comprising a biological microelectromechanical observation material;
a signal analyzer in communication with the material; and
a fluid preparation device positioned to allow fluid to flow to the sensor, wherein the fluid comprises a component from a subterranean formation.

30. The apparatus of claim 29, further comprising an additional sensor.

31. The apparatus of claim 29, further comprising an array of sensors.
